(19) United States
(12) Patent Application Publication
(10) Pub. No.: US 2024/0268764 A1
(43) Pub. Date: Aug. 15, 2024

(54) Pacing Artifact Removal

(71) Applicant: BioSig Technologies, Inc., Westport, CT (US)

(72) Inventor: Thomas G. FOXALL, Surrey (CA)

(73) Assignee: BioSig Technologies, Inc., Westport, CT (US)

(21) Appl. No.: 18/459,728

(22) Filed: Sep. 1, 2023

Related U.S. Application Data
(60) Provisional application No. 63/403,849, filed on Sep. 5, 2022.

(51) Int. Cl. A61B 5/00

(52) U.S. Cl. CPC A61B 5/7217; A61B 5/7225

(57) ABSTRACT

Disclosed herein are system, method, and computer-readable medium aspects for removing and detecting pacing artifacts from electrocardiogram and cardiac electrogram signals. An aspect includes a circuit board. The circuit board comprises two signal paths configured to receive a signal comprising cardiac data, pacing data, and background electrical noise. The first path comprises a first filter configured to amplify the cardiac data of the signal and suppress the pacing data of the signal. The second path comprises a second filter configured to amplify the pacing data of the signal and suppress the cardiac data of the signal. The circuit board also comprises a thresholding generator configured to establish a threshold amplitude value. The circuit board further comprises a thresholding module configured to apply the threshold amplitude value to the signal, thereby removing parts of amplitude of the signal that are less than or equal to the threshold amplitude value.

### CROSS-REFERENCE TO RELATED APPLICATION

This application claims benefit of provisional U.S. Patent Application No. 63/403,849, titled “Pacing Artifact Removal” filed on Sep. 5, 2022, which is incorporated herein by reference in its entirety.

### BACKGROUND

### Technical Field

Aspects of the present disclosure relate to components, systems, and methods for detecting and removing pacing artifacts from electrogram and/or electrocardiogram signals.

### Background

An electrocardiogram (ECG) is a recording of a heart's electrical activity recorded from the body's surface. An electrogram (EGM) is a recording of a heart's electrical activity recorded from within a chamber of the heart. Physicians often use ECG's to assess the health of their patients and to check for abnormalities or other conditions. The output of an ECG is a graph showing the heart's voltage over time. EGM's are used in invasive electrophysiology studies and electrophysiology treatment to determine a more localized and detailed operation of the heart in real time. The output of an EGM is also a graph showing the heart chamber's voltage over time. In cases where patients have a pacemaker installed or there is pacing from an external source, it may be difficult for a physician to distinguish between the heart's activity and the signals from the pacing source. There is often a need in the field to clarify ECG and EGM readings taken from individuals with pacemakers, or from patients undergoing procedures involving external pacing of the heart.

### SUMMARY

In aspects presented herein, electrical circuits and/or other computing devices are configured to remove and/or extract pacing artifacts from ECG and/or EGM recordings.

In an aspect, a system is disclosed for facilitating the removal of pacing artifacts from ECG and/or EGM recordings. The system can be in electronic communication with a catheter. The catheter is configured to propagate a signal comprising cardiac data, pacing data, and background electrical noise (e.g., signals that do not originate from the cardiac data or the pacing data) to the system. The system can include two signal paths. A first signal path can be configured to receive the signal. The system also comprises a first filter in the first signal path that can be configured to output a first filtered version of the signal. The first filter can be configured to amplify the cardiac data of the signal and suppress the pacing data of the signal. The system further comprises a first signal processing module in the first signal path configured to output a first processed signal. The first processed signal can be generated by squaring the first filtered version of the signal.

The system can also comprise a second filter path configured to receive the signal. The system can comprise a second filter on the second signal path that is configured to output a second filtered version of the signal. The second filter can be configured to amplify the pacing data of the signal and suppress the cardiac data of the signal. The system can further comprise a second signal processing module on the second signal path configured to output a second processed signal. The second processed signal can be generated by squaring the second filtered version of the signal.

The system can also comprise a thresholding generator configured to receive the first processed signal or the second processed signal. The thresholding generator can be further configured to establish a threshold amplitude value. The threshold amplitude value can be established based on the first processed signal or the second processed signal, or both. The system further can comprise a thresholding module that can be configured to apply the threshold amplitude value to the signal. The threshold module can be configured to remove parts of the signal having an amplitude less than the threshold amplitude value.

In another aspect, an example method is disclosed for facilitating the removal of pacing artifacts from ECG and/or EGM recordings. The method can begin by receiving a signal, wherein the signal comprises cardiac data, pacing data, and background electrical noise (e.g., signals that do not originate from the cardiac data or the pacing data). The signal can be received along a first path and a second path. A first filter in the first path can then be applied to the signal to output a first filtered version of the signal. The first filter can be configured to amplify the cardiac data of the signal and suppress the pacing data of the signal. A second filter in the second path can also be applied to the signal at the second path to output a second filtered version of the signal. The second filter can be configured to amplify the pacing data of the signal and suppress the cardiac data of the signal. Next, a threshold amplitude value can be established. Lastly, the threshold amplitude value can be applied to either the first filtered version of the signal or the second filtered version of the signal, or both. Parts of the signal having an amplitude less than or equal to the threshold amplitude value can be removed from the signal.

In yet another aspect, a non-transitory computer-readable medium is disclosed for facilitating the removal of pacing artifacts from ECG and/or EGM recordings. The non-transitory computer-readable medium has instructions stored thereon that, when executed by at least one computing device, cause the at least one computing device to perform operations. The operations can begin by receiving a signal comprising cardiac data, pacing data, and background electrical noise (e.g., signals that do not originate from the cardiac data or the pacing data). The signal can be received along a first path and a second path. A first filter in the first path can be applied to the signal to output a first filtered version of the signal. The first filter can be configured to amplify the cardiac data of the signal and suppress the pacing data of the signal. A first signal processing module can be applied to the first filtered version of the signal. The first signal processing module can be configured to square the first filtered version of the signal. A second filter in the second data path can be applied to the signal to output a second filtered version of the signal. The second filter can be configured to amplify the pacing data of the signal and suppress the cardiac data of the signal. A second signal processing module can be applied to the second filtered version of the signal. The second signal processing module can be configured to square the second filtered version of the signal. Next, the operations can establish a threshold amplitude value. The operations can then apply the threshold amplitude value to the signal such that parts of the signal having an amplitudes less than or equal to the threshold amplitude value are filtered out.

Further features and advantages, as well as the structure and operation of various aspects, are described in detail below with reference to the accompanying drawings. It is noted that the specific aspects described herein are not intended to be limiting. Such aspects are presented herein for illustrative purposes only. Additional aspects will be apparent to persons skilled in the relevant art(s) based on the teachings contained herein.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the pertinent art to make and use the disclosure.

FIG. 1 illustrates a system for facilitating the removal of pacing artifacts from ECG and/or EGM recordings in a first configuration, according to some aspects of the present disclosure.

FIG. 2 illustrates another system for facilitating the extraction of pacing signals from ECG and/or EGM recordings in a second configuration, according to some aspects of the present disclosure.

FIG. 3 is a diagram showing the establishment of a threshold amplitude value, according to some aspects of the present disclosure.

FIG. 4A is an illustration of an incoming EGM signal, according to some aspects of the present disclosure.

FIG. 4B is an illustration of a threshold amplitude value applied to an incoming EGM signal, according to some aspects of the present disclosure.

FIG. 4C is an illustration of an output of the disclosed system, according to some aspects of the present invention.

FIG. 4D is an illustration of a threshold amplitude value applied to an incoming EGM signal, according to some aspects of the present disclosure.

FIG. 4E is an illustration of an output of the disclosed system, according to some aspects of the present disclosure.

FIG. 5 is a block diagram of the threshold generator, according to some aspects of the present disclosure.

FIG. 6 is a flowchart of a method for facilitating the removal of pacing artifacts from ECG and/or EGM recordings, according to some aspects of the present disclosure.

FIG. 7 is a flowchart of a method for facilitating the extraction of pacing artifacts from ECG and/or EGM recordings, according to some aspects of the present disclosure.

FIG. 8A is a flowchart of a method for establishing a threshold amplitude value in a first configuration, according to some aspects of the present disclosure.

FIG. 8B is a flowchart of a method for establishing a threshold amplitude value in a second configuration, according to some aspects of the present disclosure.

FIG. 9 is a block diagram of an example computer system useful for implementing various aspects

In the drawings, like reference numbers generally indicate identical or similar elements. Additionally, generally, the left-most digit(s) of a reference number identifies the drawing in which the reference number first appears.

Aspects of the present disclosure will be described with reference to the accompanying drawings.

### DETAILED DESCRIPTION

It is to be appreciated that the Detailed Description section, and not any other section, is intended to be used to interpret the claims. Other sections can set forth one or more but not all exemplary aspects as contemplated by the inventor(s), and thus, are not intended to limit this disclosure or the appended claims in any way.

While this disclosure describes exemplary aspects for exemplary fields and applications, it should be understood that the disclosure is not limited thereto. Other aspects and modifications thereto are possible, and are within the scope and spirit of this disclosure. For example, and without limiting the generality of this paragraph, aspects are not limited to the software, hardware, firmware, and/or entities illustrated in the figures and/or described herein. Further, aspects (whether or not explicitly described herein) have significant utility to fields and applications beyond the examples described herein.

Aspects have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative aspects can perform functional blocks, steps, operations, methods, etc. using orderings different than those described herein.

References herein to “one aspect,” “an aspect,” “an example aspect,” or similar phrases, indicate that the aspect described can include a particular feature, structure, or characteristic, but every aspect can not necessarily include the particular feature, structure, or characteristic. Moreover, such phrases are not necessarily referring to the same aspect. Further, when a particular feature, structure, or characteristic is described in connection with an aspect, it would be within the knowledge of persons skilled in the relevant art(s) to incorporate such feature, structure, or characteristic into other aspects whether or not explicitly mentioned or described herein. Additionally, some aspects can be described using the expression “coupled” and “connected” along with their derivatives. These terms are not necessarily intended as synonyms for each other. For example, some aspects can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary aspects, but should be defined only in accordance with the following claims and their equivalents.

Provided herein are apparatus, device, system, method and/or computer-readable medium aspects, and/or combinations and sub-combinations thereof for facilitating the removal and/or extraction of pacing artifacts from ECG and/or EGM recordings.

There are several technological problems associated with facilitating the removal and/or extraction of pacing artifacts from ECG and/or EGM recordings. First, when ECG's and EGM's are performed on patients with pacemakers or during procedures where the heart is paced from an external source, the sensing electrodes will likely detect both the heart's electrical activity and the pacing electrical activity. The pacing electrical activity may not be useful for treating the patient and may lead to misdiagnosis. For example, a physician reading an ECG or EGM may mistake a pacing artifact for heart activity and report an incorrect heart rate. Second, there may be scenarios where physicians are only interested in the activity or performance of the pacemaker. In this scenario, it may be beneficial to screen out cardiac data and only present information coming from the pacemaker. Third, there may be modules or systems that automatically sense the heart rate or analyze heart function, which will count pacing activity as cardiac activity and present erroneous results to the physician

Aspects herein solve these technological problems using innovative systems and methods facilitating the removal and/or extraction of pacing artifacts from ECG and/or EGM recordings. For example, the disclosed system allows a physician to perform an ECG and/or EGM and have the pacing artifacts removed from the output of the recordings. In some aspects, the disclosed system allows for cardiac data to be removed from recordings.

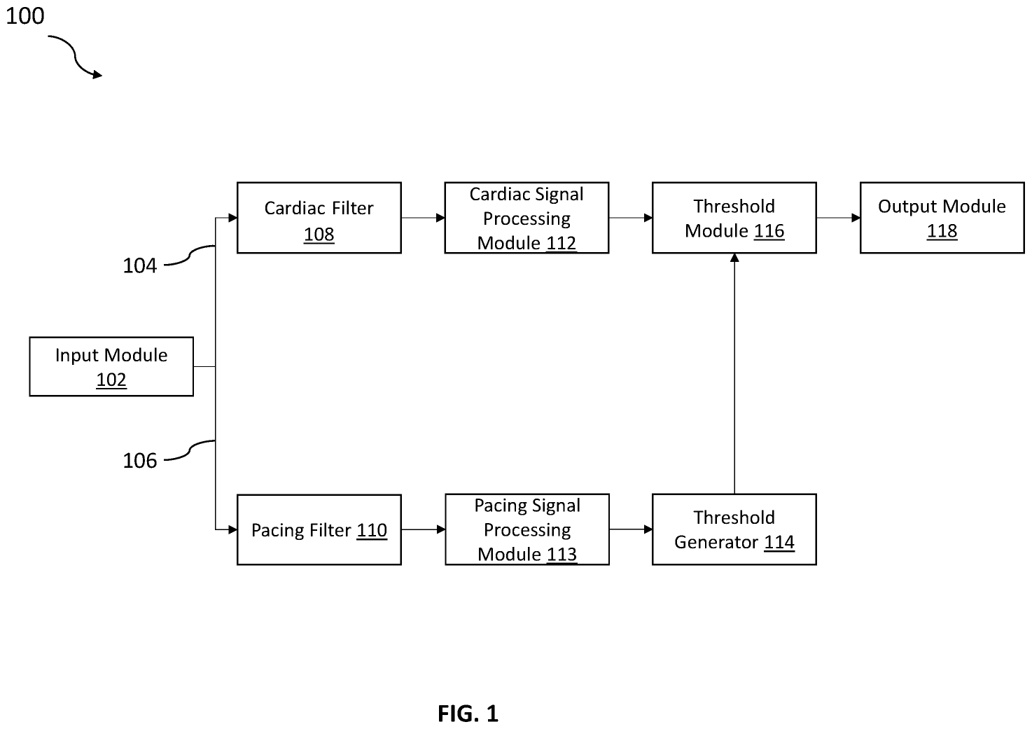

FIG. 1 is a system diagram for facilitating the removal of pacing artifacts from ECG and/or EGM recordings. System 100 can contain an input module 102, a first path 104, a second path 106, a cardiac filter 108, a pacing filter 110, a cardiac signal processing module 112, a pacing signal processing module 113, a threshold generator 114, a threshold module 116, and an output module 118.

Input module 102 can be any combination of hardware, firmware and/or software capable of receiving signals. In some aspects, input module 102 can be one or more catheters used within an EGM system. In some aspects, the catheters can be capable of detecting the electrical activity of a patient's heart. Input module 102 can contain an analog-to-digital converter configured to convert analog signals to a digital format.

Input module 102 can also be configured to receive signal samples from data stored on a digital medium. For example, data from a previously performed ECG and/or EGM may have been stored on a digital medium for future analysis. The digital medium can be a floppy disk, magnetic tape, compact disk, digital versatile disc (DVD), optical storage disk, and/any other computer data storage device. In some aspects, input module 102 can be configured to access the digital medium and play back the signals through system 100. Input module 102 can be capable of storing the received signals for later retrieval. For example, input module 102 can contain an electronic storage device where the received data can be stored. The electronic storage device can be a floppy disk, magnetic tape, compact disk, DVD, optical storage disk, and/any other computer data storage device. Input module 102 can read and/or write to the electronic storage device. It can be beneficial to save the signal data sent to input module 102 for future analysis. In some aspects, the signals received by input module 102 can originate from a human heart with a pacemaker or with pacing externally supplied. In this scenario, the signals can contain both cardiac data and pacing data. Input module 102 can forward signals received from the catheter to a first path 104 and a second path 106.

First path 104 and second path 106 can be any electrical circuit capable of receiving and propagating a signal received from input module 102. In some aspects, first path 104 and second path 106 can be made of a conductive material (e.g., copper wire, or the like). In some aspects, first path 104 and second path 106 can be implemented as logical paths in a software program. First path 104 can forward the received signal to a cardiac filter 108. Second path 106 can forward the received signal to pacing filter 110.

Cardiac filter 108 can be any combination of hardware, firmware, and/or software capable of filtering parts of the signal. Cardiac filter 108 can contain one or more filters applied in series. In some aspects, cardiac filter 108 can first apply a bandpass filter to suppress baseline wander, powerline interference, far field signals, and other high frequency noise as would be appreciated by a person of ordinary skill in the art. Cardiac filter 108 can also contain a lowpass filter designed to remove or suppress unwanted high frequency signals. Cardiac filter 108 can be configured to pass cardiac signals of interest while filtering out pacing and other signals such as background electrical noise (e.g., signals that do not originate from the cardiac data or the pacing data). Signals can be exchanged between the cardiac filter 108 and the cardiac signal processing module 112.

Cardiac signal processing module 112 and pacing signal processing module 113 can be any combination of hardware, firmware, and/or software capable of manipulating an input signal. Cardiac signal processing module 112 and pacing signal processing module 113 can contain one or more computer processors connected to a communication infrastructure or bus. In some aspects, the one or more computer processors may each be a graphics processing unit (GPU). In some aspects, a GPU is a specialized electronic circuit designed to process mathematically intensive operations. The GPU can have a parallel structure that is efficient for parallel processing of large blocks of data, such as mathematically intensive data common to computer graphics applications, images, videos, etc. In some aspects, the one or more computer processors can each be a digital signal processor (DSP). In some aspects, a DSP is a specialized electronic circuit designed to process mathematically intensive operations. The DSP can have a parallel structure that is efficient for parallel processing of large blocks of data, such as mathematically intensive data common to computer signal processing applications.

Cardiac signal processing module 112 and pacing signal processing module 113 can also contain memory, such as random access memory (RAM). The memory can have control logic (e.g., computer software) and/or data stored therein. As would be appreciated by a person of ordinary skill in the art (POSA), manipulating a signal may involve a mathematical algorithm that takes one or more signal samples as input, processes them, and produces one or more potentially modified signal samples as output. In some aspects, cardiac signal processing module 112 and pacing signal processing module 113 can be capable of receiving an input signal, squaring the signal such that all amplitude values become positive, and transmitting the resulting signal.

Pacing filter 110 can be any combination of hardware, firmware, and/or software capable of removing parts of a signal. Pacing filter 110 can contain multiple filters applied in series, for example. In some aspects, pacing filter 110 can be a high pass filter that selectively allows high frequency pacing spikes through while filtering out lower frequency cardiac signals. Pacing filter 110 can output a processed pacing signal that can be sent to pacing signal processing module 113.

Threshold generator 114 can be any combination of hardware, firmware, and/or software capable of receiving a signal output from cardiac signal processing module 112 and/or pacing signal processing module 113. Threshold generator 114 can contain one or more computer processors connected to a communication infrastructure or bus. In some aspects, the one or more processors may each be a GPU. In some aspects, a GPU is a specialized electronic circuit designed to process mathematically intensive operations. The GPU can have a parallel structure that is efficient for parallel processing of large blocks of data, such as mathematically intensive data common to computer graphics applications, images, videos, etc. In some aspects, threshold generator 114 can be DSP. In some aspects, a DSP is a specialized electronic circuit designed to process mathematically intensive operations. The DSP can have a parallel structure that is efficient for parallel processing of large blocks of data, such as mathematically intensive data common to signal processing applications. Threshold generator 114 can also contain memory, such as RAM. The memory can have control logic (e.g., computer software) and/or data stored therein.

In some aspects, threshold generator 114 can use the processed pacing signal data to establish a threshold amplitude value. Threshold generator 114 can establish the threshold amplitude value from the amplitude and/or shape of the processed pacing signal data. Threshold generator 114 can adjust the threshold amplitude value. For example, if the amplitude of the processed pacing signal suddenly increases, threshold generator 114 can detect this change and update the threshold amplitude value. Threshold generator 114 can automatically update the threshold amplitude value after it has received a predetermined number of signals, or a certain amount of time has passed. Threshold generator 114 can also update the threshold amplitude value if new signal data is different from the current signal data. Threshold generator 114 can analyze the processed pacing signal data in order to determine an average amplitude and/or an average shape. In some aspects, the established threshold amplitude value may encapsulate the patient's pacing signal data but not the patient's cardiac data. The threshold generator 114 can send the threshold amplitude value to the threshold module 116.

Threshold module 116 can be any combination of hardware, firmware, and/or software configured to apply the established threshold amplitude value to the output of cardiac signal processing module 112. Threshold module 116 can contain one or more computer processors connected to a communication infrastructure or bus. In one embodiment, the one or more computer processors may each be a GPU In some aspects, a GPU is a specialized electronic circuit designed to process mathematically intensive operations. The GPU can have a parallel structure that is efficient for parallel processing of large blocks of data, such as mathematically intensive data common to computer graphics applications, images, videos, etc. In some aspects, threshold module 116 can be a DSP. In some aspects, a DSP is a specialized electronic circuit designed to process mathematically intensive operations. The DSP can have a parallel structure that is efficient for parallel processing of large blocks of data, such as mathematically intensive data common to signal processing applications. Threshold module 116 can also contain memory, such as random access memory (RAM). The memory can have control logic (i.e. computer software) and/or data stored therein

Threshold module 116 can take as input the processed cardiac data from cardiac signal processing module 112 and the threshold amplitude value. Threshold module 116 can then compare the threshold amplitude value to the processed cardiac data. Threshold module 116 can filter parts of the processed cardiac data having an amplitude greater than the threshold amplitude value to create an altered signal. Threshold module 116 can then send the altered signal to output module 118.

Output module 118 can be any combination of hardware, firmware, and/or software capable of outputting the altered signal. In one embodiment, output module 118 can be a screen that displays the altered signal. For example, a physician performing an ECG or EGM on a patient may view the output from output module 118 in order to perform a medical assessment on the patient. Output module 118 can label the threshold amplitude value and the ECG or EGM signal data so that a physician can distinguish between the two components. In some aspects, output module 118 can assign the threshold amplitude value and the ECG or EGM signal data different colors so that a physician can distinguish between the patient's data and the threshold amplitude value. In some aspects, output module 118 can be software to record the signal to a digital medium for future analysis. The digital medium device can be a floppy disk, magnetic tape, compact disk, DVD, optical storage disk, and/any other computer data storage device

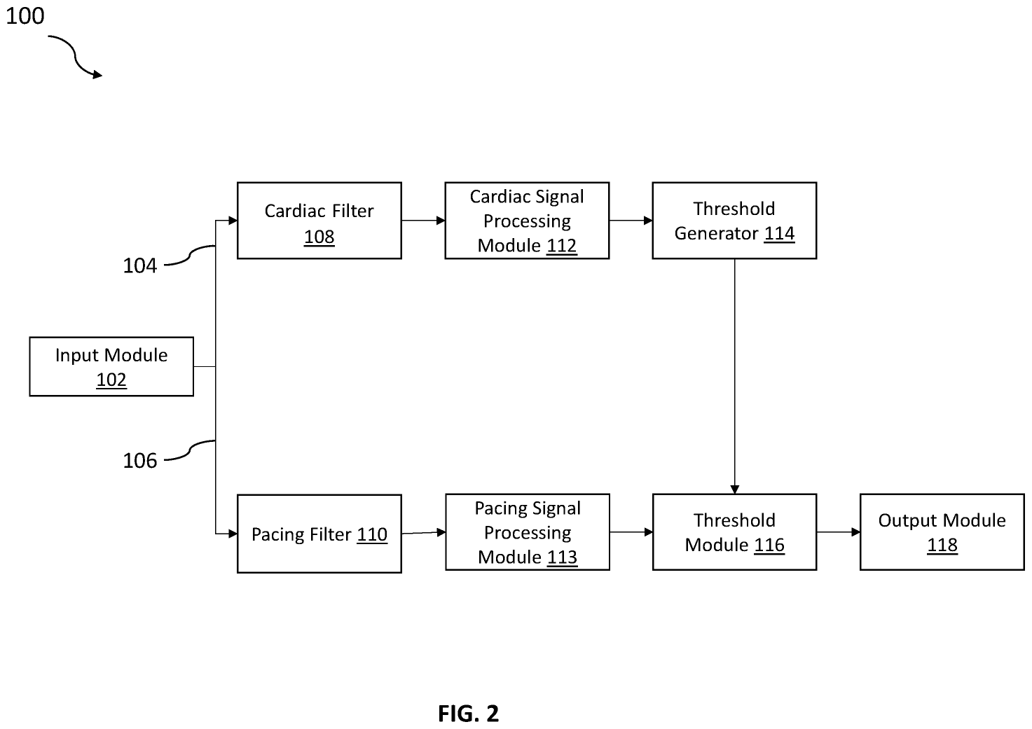

FIG. 2 is a block diagram showing the flow paths of an electrical signal through system 100, according to some aspects of the present disclosure. As depicted in FIG. 2, threshold generator 114 and threshold module 116 can be swapped such that threshold generator 114 is on first path 104 and threshold module 116 is on second path 106. In some aspects, the cardiac signals may be used by threshold generator 108 to establish the threshold amplitude value in order to remove cardiac signals instead of pacing signals. This approach may be advantageous in a scenario where a physician or other party is interested in analyzing or identifying pacing artifacts as opposed to cardiac data. For example, the identified pacing artifacts can then be used as locators to identify segments containing pacing signals in the original ECG and/or EGM data. These identified segments can then be reconstructed by interpolating the cardiac data in the segments containing the pacing artifact.

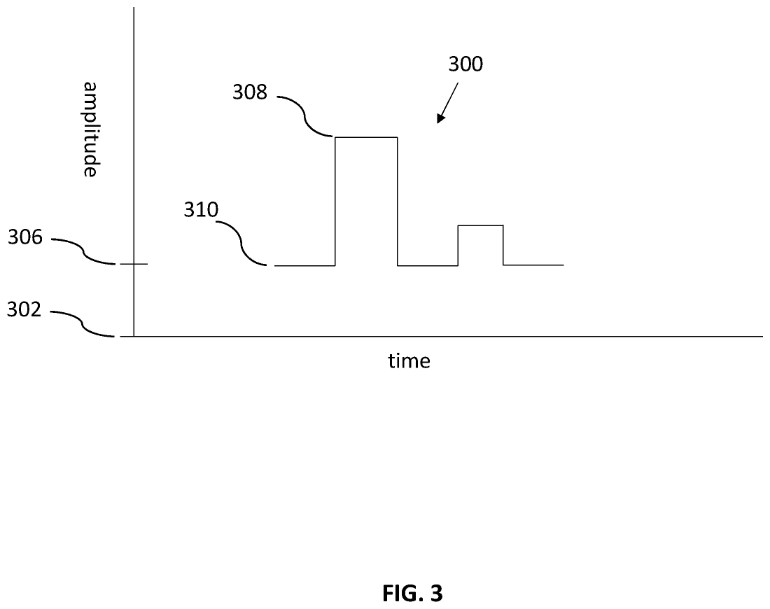

FIG. 3 illustrates establishment of a threshold amplitude value 300 by threshold generator 114, according to some aspect of the present disclosure. Threshold amplitude value 300 can consist of a maximum amplitude value 308 and a minimum amplitude value 310. In some aspects, maximum amplitude value 308 and minimum amplitude value 310 can be expressed in millivolts. Maximum amplitude value 308 can be defined as a value greater than or equal to minimum amplitude value 310. Minimum amplitude value 310 can be defined as a value greater than or equal to a base threshold amplitude value 306. Base threshold amplitude value 306 can be defined as a value greater than or equal to a threshold amplitude floor 302. In some aspects, threshold amplitude floor 302 can be expressed as 0 millivolts. Base threshold amplitude value 306 can have a constant value that does not change during the operation of system 100. Base threshold amplitude value 306 can have a variable value that is a function of incoming processed data. Threshold amplitude value 300 can have threshold amplitude values that change over time.

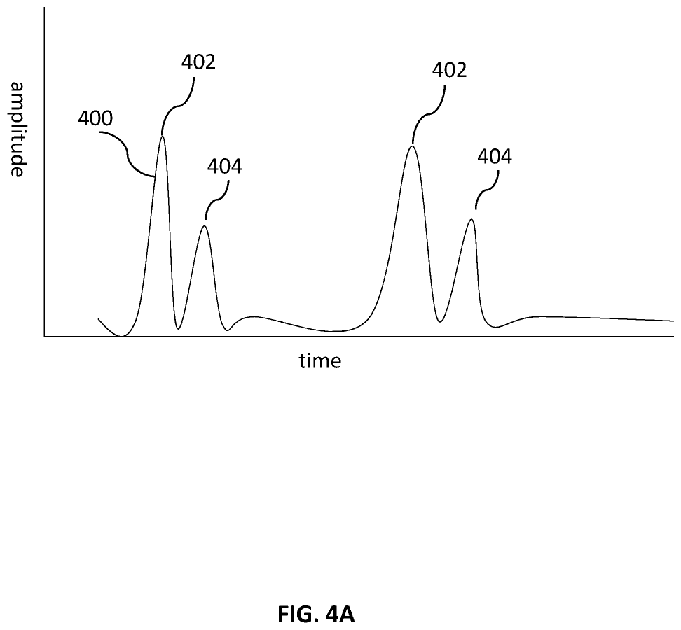

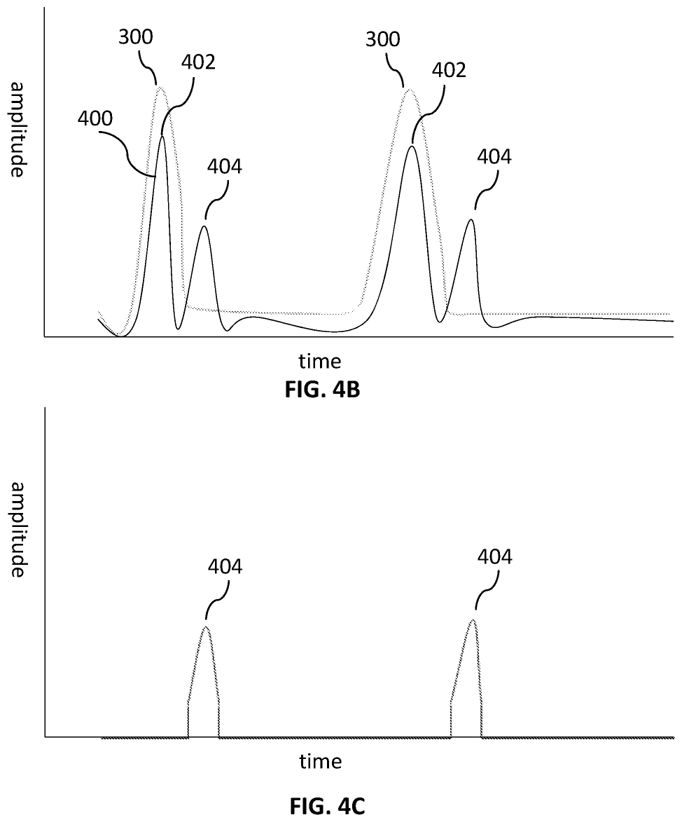

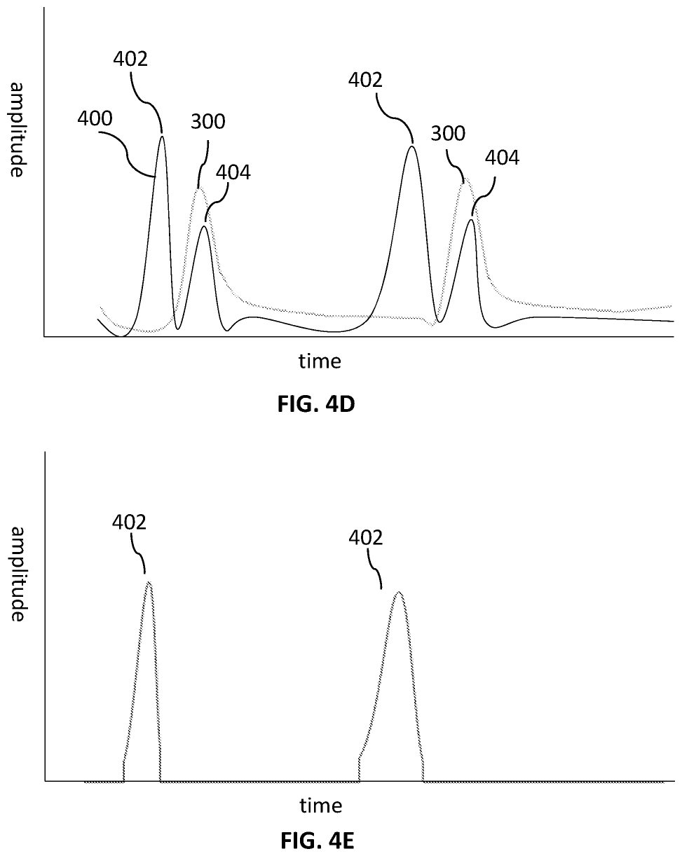

FIG. 4A is an illustration of an example EGM 400. EGM 400 can contain pacing artifacts 402 and intracardiac data 404, according to some aspect of the present disclosure.

FIG. 4B is an illustration of system 100 where threshold 300 is applied to EGM 400 according to some aspect of the present disclosure. FIG. 4B is discussed with reference to FIG. 3. In some aspects, threshold amplitude value 300 is shown covering pacing artifacts 402. In some aspects, only intracardiac data 404 is able to exceed the value of the threshold amplitude value 300 at any point in the EGM segment shown.

FIG. 4C is an illustration of the output of system 100 where threshold amplitude value 300 has been applied to EGM 400 according to some aspect of the present disclosure. FIG. 4B is discussed with reference to FIG. 3. In some aspects, once threshold amplitude value 300 has been applied to EGM 400, pacing artifacts 402 are removed and only the intracardiac data peaks 404 remain. This may be beneficial in a scenario where a physician only wants to view intracardiac data peaks 404 or where this data goes to another module that analyzes heart operation.

FIG. 4D is an illustration of system 100 where threshold amplitude value 300 has been applied to EGM 400, according to some aspects of the present disclosure. In some aspects, threshold amplitude value 300 is shown covering intracardiac data 404, according to some aspects of the present disclosure. In this case, only pacing artifacts 402 are able to exceed the value of the threshold amplitude value at any point in the EGM segment shown.

FIG. 4E is an illustration of the output of system 100 where threshold amplitude value 300 has been applied to EGM 400, according to some aspects of the present disclosure. In some aspects, threshold amplitude value 300 has been applied to intracardiac data 404 and only pacing artifacts 402 remain. This output may be beneficial if a physician is interested in marking pace signal positions or if the output goes to another module that uses the information to interpolate cardiac data during the pacing artifact duration.

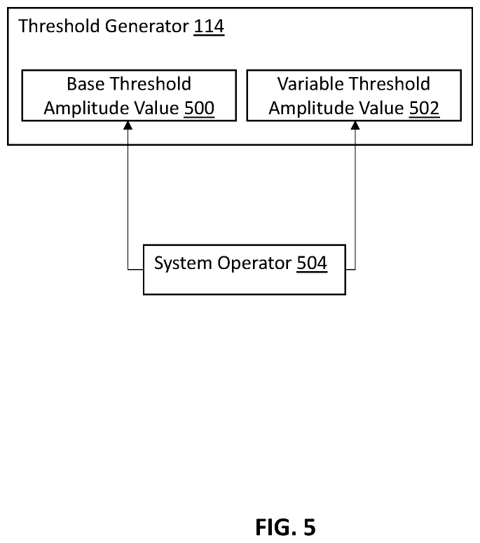

FIG. 5 is a block diagram of threshold generator 114, according to some aspects of the present disclosure. Threshold generator 114 can comprise two subcomponents, a base threshold value block 500 and a variable threshold value block 502. In some aspects, base threshold amplitude value block 500 can be set at a constant value or as a functional value dependent on the state of the incoming signal data. Parts of a signal with amplitude less than base threshold amplitude value 500 can be removed. This may be beneficial in order to remove unwanted signals, noise and other interference from the incoming signal data. Variable threshold amplitude value 502 can consist of an amplitude and a shape. Variable threshold amplitude value 502 can be constructed from pacing data or cardiac data within an ECG or EGM. An operator 504 can be able to interface with threshold generator 114 and alter the base threshold amplitude value 500 and/or variable threshold amplitude value 502. Base threshold amplitude value 500 and variable threshold amplitude value 502 can be independent from one another such that a change in one has no effect upon the other. Base threshold amplitude value 500 and variable threshold amplitude value 502 can be combined and sent to threshold module 116.

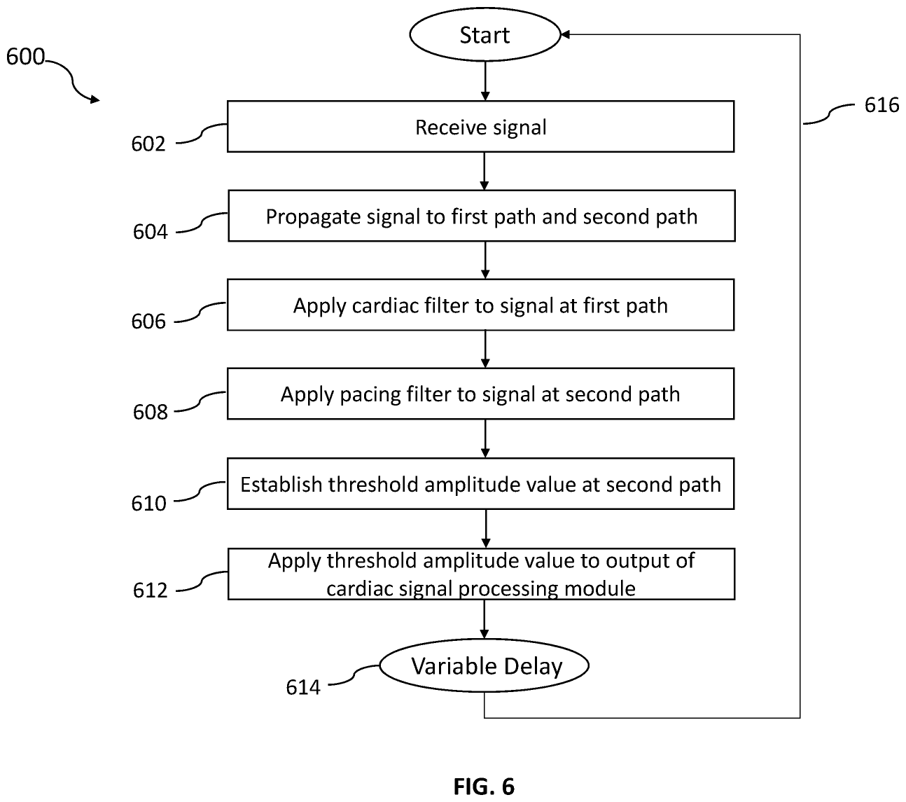

FIG. 6 is a flowchart for method 600 for facilitating the removal of pacing artifacts from ECG and/or EGM recordings, according to some aspects of the present disclosure. It is to be appreciated that not all steps can be needed to perform the disclosure provided herein. Further, some of the steps can be performed simultaneously, or in a different order than shown in FIG. 6, as would be understood by a person of ordinary skill in the art.

At step 602, a signal is received. The signal may be historical data read from a file or live data collected by a catheter connected to a patient.

At step 604, the signal can be propagated to a first path and a second path. The first and second paths can be physically independent from one another. In some aspects, such as a software implementation, the first and second paths can be logically independent.

At step 606, a cardiac filter and signal processing module are applied to the signal at the first path. The cardiac filter can be configured to suppress pacing data within the received signal and enhance cardiac data (e.g., reduce or filter out frequencies outside of the frequency range of the received signal). The signal processing module can be configured to square the frequencies of the signal at the first path.

At step 608, a pacing filter and signal processing module are applied to the signal at the second path. The pacing filter can be configured to suppress cardiac data within the received signal and enhance pacing data (e.g., reduce or filter out frequencies outside of the frequency range of the received signal). The signal processing module can be configured to square the frequencies of the signal at the second path.

At step 610, a threshold amplitude value is established at the second path. The threshold amplitude value can be established based on the pacing data in the signal after the application of the pacing filter and signal processing module. Establishing the threshold amplitude value after reducing cardiac signals helps insure that the threshold amplitude value will not increase due to characteristics of the cardiac data.

At step 612, the threshold amplitude value can be applied to the output of the cardiac signal processing module. In some aspects, the threshold amplitude value removes parts of the signal that have an amplitude less than the threshold amplitude value. In some aspects, the output contains only the cardiac data.

At step 614, a variable delay can be introduced. The length of the variable delay may be changed based upon the application. The variable delay may allow the system align the output in time with other system data having different processing delay times.

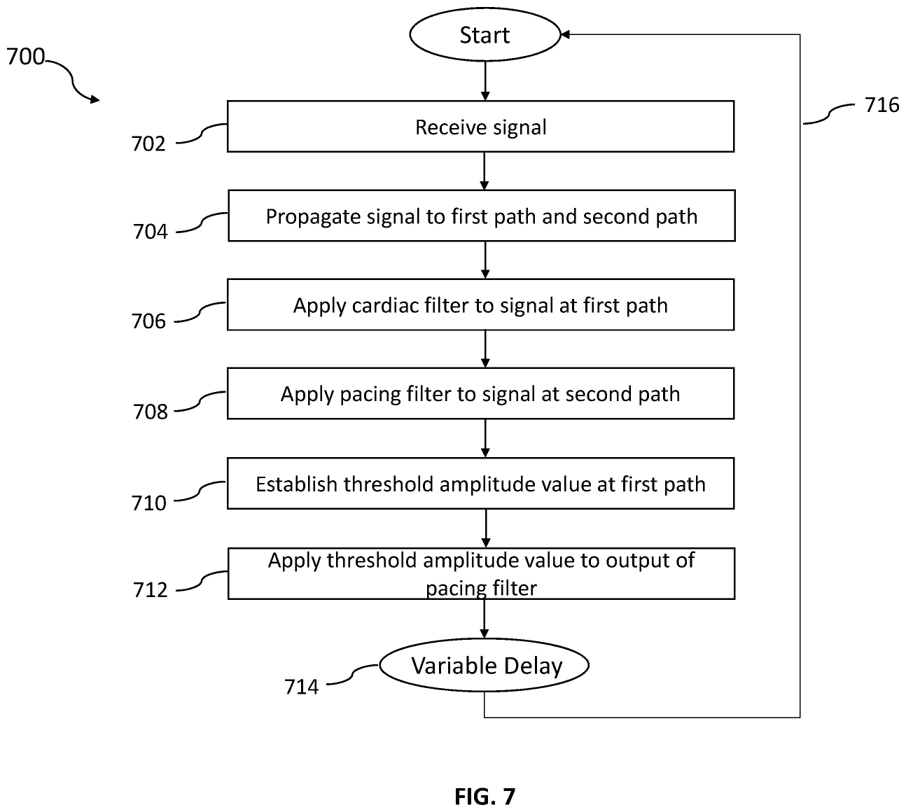

FIG. 7 is a flowchart for method 700 for facilitating the extraction of pacing artifacts from ECG and/or EGM recordings, according to some aspects of the present disclosure. It is to be appreciated that not all steps can be needed to perform the disclosure provided herein. Further, some of the steps can be performed simultaneously, or in a different order than shown in FIG. 7, as would be understood by a person of ordinary skill in the art.

At step 702, a signal can be received. The signal can be historical data read from a file or live data collected by a catheter connected to a patient.

At step 704, the signal can be propagated to a first path and a second path. The first and second paths can be physically independent from one another. In some aspects, such as a software implementation, the first and second paths can be logically independent.

At step 706, a cardiac filter and signal processing module can be applied to the signal at the first path. The cardiac filter can be configured to reduce pacing data within the received signal. The signal processing module can be configured to square the frequencies of the signal at the first path.

At step 708, a pacing filter can be applied to the signal at the second path. The pacing filter can be configured to reduce cardiac data within the received signal.

At step 710, a threshold amplitude value can be established at the first path. The threshold amplitude value can be established based on the cardiac data in the signal after the application of the cardiac filter and signal processing module. Establishing the threshold amplitude value after reducing pacing signals helps insure that the threshold amplitude value will not increase due to characteristics of the pacing data.

At step 712 the threshold amplitude value can be applied to the output of the pacing filter. In some aspects, the threshold amplitude value removes parts of the signal that have an amplitude less than threshold amplitude value. In some aspects, the output contains only the pacing data.

At step 714, a variable delay can be introduced. The length of the variable delay can be changed based upon the application. The variable delay can allow the system to align the output in time with other system data having different processing delay times.

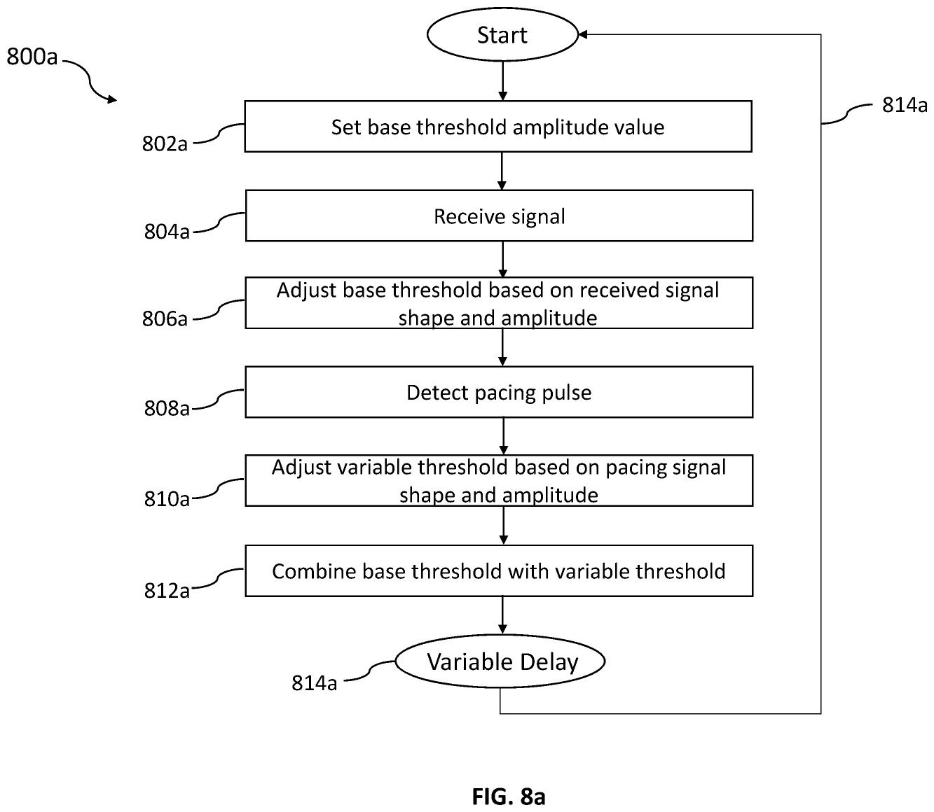

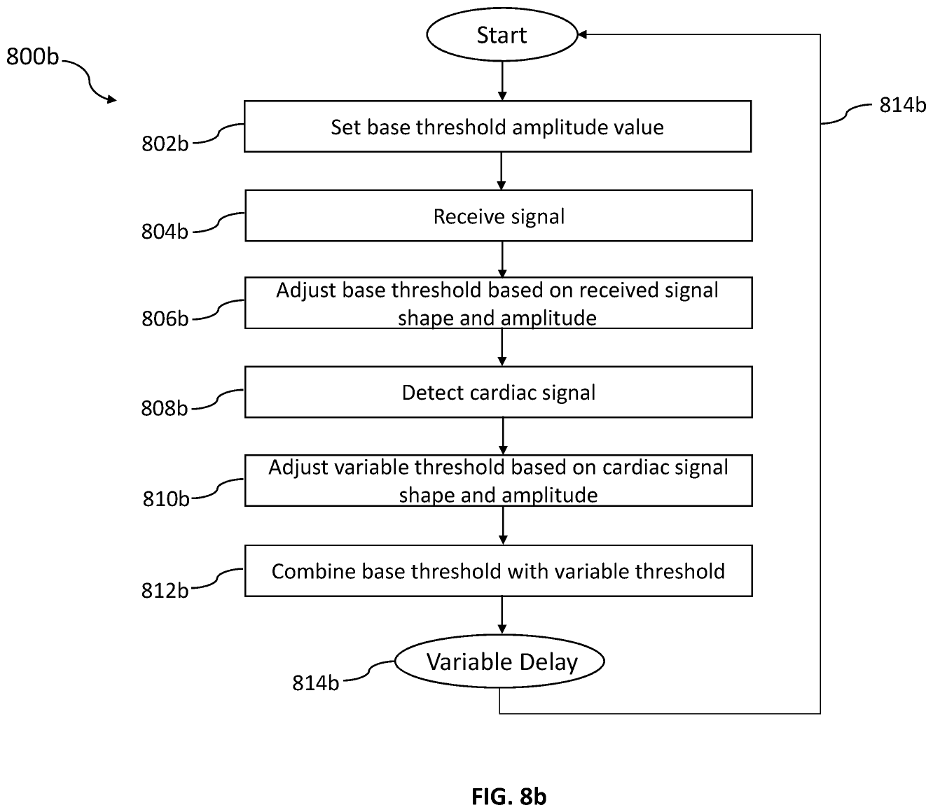

FIG. 8A is a flowchart for method 800a for establishing a threshold amplitude value, according to some aspects of the present disclosure. It is to be appreciated that not all steps can be needed to perform the disclosure provided herein. Further, some of the steps can be performed simultaneously, or in a different order than shown in FIG. 8, as would be understood by a person of ordinary skill in the art.

Method 800a can be implemented by hardware such as an integrated circuit or in a computing device such as a desktop computer. However, method 800a is not limited to those example aspects.

In 802a, a base threshold amplitude value can be set. The base threshold amplitude value can have a default value or minimum value that is used each time the system is used. In some aspects, a user or operator may be able to define and/or update the base threshold amplitude value.

In 804a, a signal can be received. The signal can contain live data. For example, the signal can be the output of an ECG or EGM monitoring system connected to a patient. In some aspects, the signal may be in the form of recorded data stored within a readable medium.

In 806a, the base threshold amplitude value can be adjusted based on the shape and amplitude of the received signal. The adjusted threshold shape may be rectangular, triangular, raised cosine, or any other shape as would be appreciated by a POSA.

In 808a, a pacing pulse can be detected within the signal data. The pacing pulse can be correlated with activity from a pacemaker within the patient or from an external source.

In 810a, the variable threshold amplitude value can be adjusted based on the shape and amplitude of the pacing pulse. The adjusted threshold shape may be rectangular, triangular, raised cosine, or any other shape as would be appreciated by a POSA. The effect of the pacing pulse can be to increase the threshold amplitude value in regions where there is pacing, so that any remaining pacing signals in the cardiac data stream cannot exceed the threshold amplitude value.

In 812a, the base threshold value and variable threshold value can be combined. The resulting threshold amplitude value can then be used to remove pacing artifacts from ECG and/or EGM output.

In 814a, a variable delay can be introduced. The length of the variable delay can be changed based upon the application. The variable delay can allow the system to align the output in time with other system data having different processing delay times.

FIG. 8B is a flowchart for method 800b for generating a threshold, according to some aspects of the present disclosure. It is to be appreciated that not all steps can be needed to perform the disclosure provided herein. Further, some of the steps can be performed simultaneously, or in a different order than shown in FIG. 8B, as would be understood by a person of ordinary skill in the art.

Method 800b can be implemented by hardware such as an integrated circuit or in a computing device such as a desktop computer. However, method 800b is not limited to those example aspects.

In 802b, a base threshold amplitude value can be set. The base threshold amplitude value can have a default value or minimum value that is used each time the system is used. In some aspects, a user or operator may be able to define and/or update the base threshold value.

In 804b, a signal can be received. The signal can contain live data. For example, the signal can be the output of an ECG and/or EGM monitoring system connected to a patient. In some aspects, the signal may be in the form of recorded data stored within a readable medium.

In 806b, the base threshold amplitude value can be adjusted based on the shape and amplitude of the received signal. The adjusted threshold shape may be rectangular, triangular, raised cosine, or any other shape as would be appreciated by a POSA.

In 808b, a cardiac pulse can be detected within the signal data. The cardiac signal can be correlated with activity from the heart of the patient.

In 810b, the variable threshold amplitude value can be adjusted based on the shape and amplitude of the cardiac pulse. The adjusted threshold shape may be rectangular, triangular, raised cosine, or any other shape as would be appreciated by a POSA. The effect of the cardiac pulse can be to increase the threshold amplitude value in regions where there is cardiac signal, so that any remaining cardiac signals in the pacing data stream cannot exceed the threshold amplitude value.

In 812b, the base threshold amplitude value and variable threshold amplitude value can be combined. The resulting threshold amplitude value can then be used to detect pacing artifacts from ECG and/or EGM output.

In 814b, a variable delay can be introduced. The length of the variable delay can be changed based upon the application. The variable delay can allow the system align the output in time with other system data having different processing delay times.

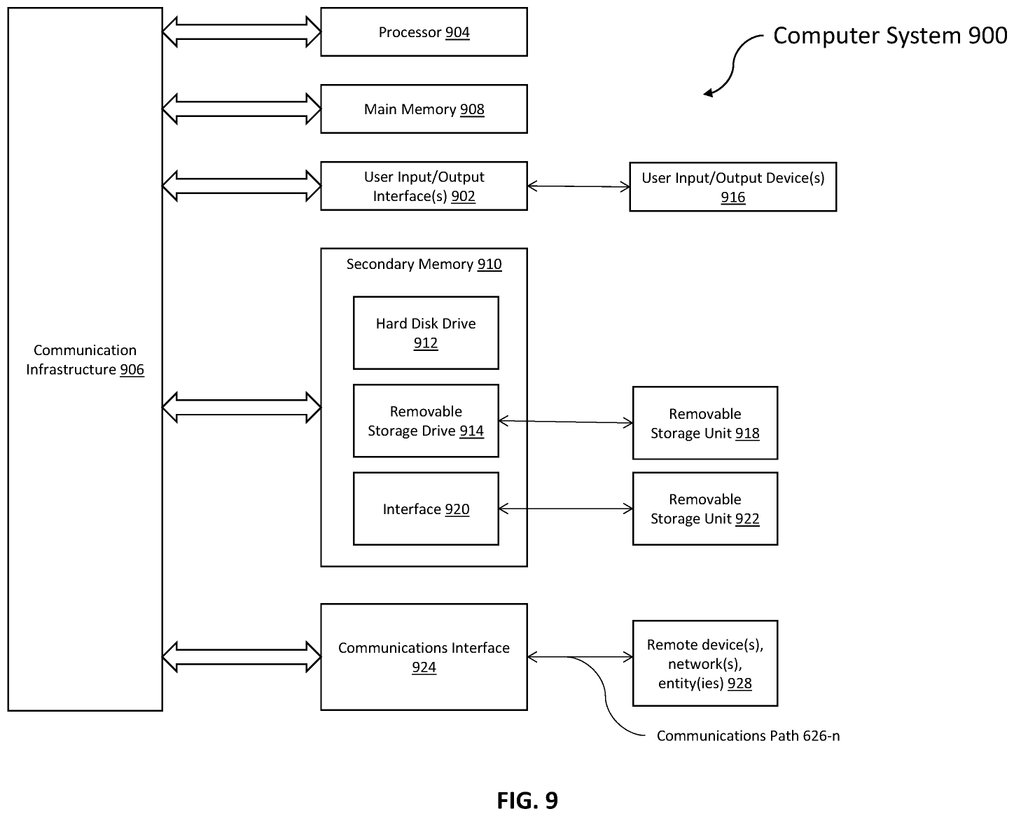

Various aspects can be implemented, for example, using one or more computer systems, such as computer system 900 shown in FIG. 9. Computer system 900 can be used, for example, to implement a system for facilitating the removal of pacing artifacts from ECG and/or EGM recordings. For example, computer system 900 can receive one or more signals, apply one or more filters to the signals, establish a threshold amplitude value, and apply the threshold amplitude value to the one or more signals. Computer system 900 can be any computer capable of performing the functions described herein.

Computer system 900 can be any well-known computer capable of performing the functions described herein.

Computer system 900 includes one or more processors (also called central processing units, or CPUs), such as a processor 904. Processor 904 is connected to a communication infrastructure or bus 906. Processor 904 can be used to manipulate signals input to computer system 900. In one embodiment, processor 904 can apply one or more filters to the signals. In another embodiment, processor 904 can square the frequencies of the incoming signals.

One or more processors 904 may each be a graphics processing unit (GPU). In an aspect, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc. One or more processors 904 may each also be a digital signal processor (DSP). In an aspect, a DSP is a processor that is a specialized electronic circuit designed to process mathematically intensive applications. The DSP may have a parallel structure that is efficient for parallel processing of large blocks of data, such as mathematically intensive data common to computer signal processing applications.

Computer system 900 also includes user input/output device(s) 916, such as monitors, keyboards, pointing devices, etc., that communicate with communication infrastructure 906 through user input/output interface(s) 902. Computer system 900 can use input/output device(s) 916 to receive one or more signals. Input/output device(s) 916 can also be used to display the output of computer system 900 on a screen.

Computer system 900 also includes a main or primary memory 908, such as random access memory (RAM). Main memory 908 may include one or more levels of cache. Main memory 908 has stored therein control logic (i.e., computer software) and/or data.

Computer system 900 may also include one or more secondary storage devices or memory 910. Secondary memory 910 may include, for example, a hard disk drive 912 and/or a removable storage device or drive 914. Removable storage drive 914 may be a floppy disk drive, a magnetic tape drive, a compact disk drive, an optical storage device, tape backup device, and/or any other storage device/drive. Secondary memory 910 may be used to record incoming signals for future analysis.

Removable storage drive 914 may interact with a removable storage unit 918. Removable storage unit 918 includes a computer usable or readable storage device having stored thereon computer software (control logic) and/or data. Removable storage unit 918 may be a floppy disk, magnetic tape, compact disk, DVD, optical storage disk, and/any other computer data storage device. Removable storage drive 914 reads from and/or writes to removable storage unit 918 in a well-known manner.

According to an exemplary aspect, secondary memory 910 may include other means, instrumentalities or other approaches for allowing computer programs and/or other instructions and/or data to be accessed by computer system 900. Such means, instrumentalities or other approaches may include, for example, a removable storage unit 922 and an interface 920. Examples of the removable storage unit 922 and the interface 920 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface. Removable storage unit 922 and interface 920 can be used to input previously recorded signals to computer system 900.

Computer system 900 may further include a communication or network interface 924. Communication interface 924 enables computer system 900 to communicate and interact with any combination of remote devices, remote networks, remote entities, etc. (individually and collectively referenced by reference number 928). For example, communication interface 924 may allow computer system 900 to communicate with remote devices 928 over communications path 926, which may be wired and/or wireless, and which may include any combination of LANs, WANs, the Internet, etc. Control logic and/or data may be transmitted to and from computer system 900 via communication path 926.

In an aspect, a tangible, non-transitory apparatus or article of manufacture comprising a tangible, non-transitory computer useable or readable medium having control logic (software) stored thereon is also referred to herein as a computer program product or program storage device. This includes, but is not limited to, computer system 900, main memory 908, secondary memory 910, and removable storage units 918 and 922, as well as tangible articles of manufacture embodying any combination of the foregoing. Such control logic, when executed by one or more data processing devices (such as computer system 900), causes such data processing devices to operate as described herein, such as for removing pacing signal artifacts, or the like filtering, as described herein.

Based on the teachings contained in this disclosure, it will be apparent to persons skilled in the relevant art(s) how to make and use aspects of this disclosure using data processing devices, computer systems and/or computer architectures other than that shown in FIG. 9. In particular, aspects can operate with software, hardware, and/or operating system implementations other than those described herein.

What is claimed is:

1. A method for removing or extracting pacing signal artifacts, comprising:
receiving a signal comprising cardiac data, pacing data, and background electrical noise at a first path and a second path, wherein the background electrical noise comprises signals that do not originate from the cardiac data or the pacing data;
first filtering the signal, using a first filter in the first path, thereby outputting a first filtered version of the signal, wherein the first filtering amplifies the cardiac data of the signal and suppresses the pacing data of the signal;
second filtering the signal, using a second filter in the second path, thereby outputting a second filtered version of the signal, wherein the second filtering amplifies the pacing data of the signal and suppresses the cardiac data of the signal;
establishing a threshold amplitude value; and
applying the threshold amplitude value to the first filtered version of the signal or the second filtered version of the signal to output a thresholded signal, wherein parts of amplitude of the signal that are less than or equal to the threshold amplitude value are removed from the thresholded signal.

2. The method of claim 1, wherein the threshold amplitude value is applied to the first filtered version of the signal.

3. The method of claim 1, wherein the threshold amplitude value is applied to the second filtered version of the signal.

4. The method of claim 1, wherein the first filter is a low frequency bandpass filter.

5. The method of claim 1, wherein the second filer is a high frequency pass filter.

6. The method of claim 1, wherein the threshold amplitude value comprises:
a first parameter, wherein the first parameter has a constant value; and
a second parameter, and
wherein the method further comprises adjusting the second parameter based on an amplitude of the pacing data within the signal.

7. The method of claim 1, wherein the threshold amplitude value comprises:
a first parameter, wherein the first parameter has a constant value; and
a second parameter, and
wherein the method further comprises adjusting the second parameter based on an amplitude of the cardiac data within the signal.

8. The method of claim 1, wherein the method is performed by a computer comprising one or more processors.

9. A system for removing pacing signal artifacts from a signal, the signal having cardiac data, pacing data, and background electrical noise, wherein the background electrical noise comprises signals that do not originate from the cardiac data or the pacing data, the system comprising:
a first signal path, configured to receive the signal, comprising:
a first filter configured to amplify the cardiac data of the signal and suppress the pacing data of the signal and output a first filtered version of the signal; and
a first signal processing module configured to receive and square the first filtered version of the signal and output a first processed signal;
a second filter path configured to receive the signal, comprising:
a second filter configured to amplify pacing data of the signal and suppress the cardiac data of the signal and to output a second filtered version;
a second signal processing module configured to receive and square the second filtered version of the signal and output a second processed signal;
a thresholding generator configured to receive the first processed signal or the second processed signal and establish a threshold amplitude value; and
a thresholding module configured to apply the threshold amplitude value to the signal and output a thresholded signal, wherein parts of amplitude of the signal that are less than the threshold amplitude value are removed from the thresholded signal.

10. The system of claim 9, wherein the threshold module is configured to apply the threshold amplitude value to the first processed signal.

11. The system of claim 9, wherein the threshold module is configured to apply the threshold amplitude value to the second processed signal.

12. The system of claim 9, wherein the first filter is a low frequency bandpass filter.

13. The system of claim 9, wherein the second filter is a high frequency pass filter.

14. The system of claim 9, wherein the threshold amplitude value comprises:
a first parameter, wherein the first parameter has a constant value;
a second parameter, wherein the second parameter is adjusted based on the amplitude of the pacing data or cardiac data within the cardiac signal; and
a third parameter, wherein the third parameter corresponds to a shape of the first processed signal or the second processed signal.

15. The system of claim 9, wherein the system is configured to receive operator input to modify the first parameter, the second parameter, or the third parameter.

16. A non-transitory computer-readable medium having instructions stored thereon that, when executed by one or more computing devices, cause the one or more computing devices to perform operations comprising:
receiving a signal comprising cardiac data, pacing data, and background electrical noise at a first path and a second path, wherein the background electrical noise comprises signals that do not originate from the cardiac data or the pacing data;
first filtering the signal, using a first filter in the first path, thereby outputting a first filtered version of the signal, wherein the first filtering amplifies the cardiac data of the signal and suppresses the pacing data of the signal;
applying first signal processing to the first filtered version of the signal, wherein the first signal processing squares the first filtered version of the signal;
second filtering the signal, using a second filter in the second path, thereby outputting a second filtered version of the signal, wherein the second filtering amplifies the pacing data of the signal and suppresses the cardiac data of the signal;
applying a second signal processing to the second filtered version of the signal, wherein the second signal processing squares the second filtered version of the signal;
establishing a threshold amplitude value; and
applying the threshold amplitude value to the signal to output a thresholded signal,
wherein parts of amplitude of the signal that are less than or equal to the threshold amplitude value are removed from the thresholded signal.

17. The non-transitory computer-readable medium claim 16, wherein the threshold amplitude value is applied to the first filtered version of the signal.

18. The non-transitory computer-readable medium of claim 16, wherein the threshold amplitude value is applied to the second filtered version of the signal.

19. The non-transitory computer-readable medium of claim 16, wherein the first filter is a low frequency bandpass filter, and the second filter is a high frequency pass filter.

20. The non-transitory computer-readable medium of claim 16, wherein the threshold amplitude value comprises:
a first parameter, wherein the first parameter has a constant value; and
a second parameter, and
wherein the operations further comprise adjusting the second parameter based on the amplitude of the pacing data within the signal.
